(19) United States
(12) Patent Application Publication
(10) Pub. No.: US 2024/0165013 A1
(43) Pub. Date: May 23, 2024

(54) COSMETIC COMPOSITION

(71) Applicant: GIVAUDAN SA, Vernier (CH)

(72) Inventors: Amandine SCANDOLERA, Reims (FR); Romain REYNAUD, Toulouse (FR)

(21) Appl. No.: 18/282,406

(22) PCT Filed: Mar. 16, 2022
(86) PCT No.: PCT/EP2022/056783

§ 371 Date: Sep. 15, 2023

(30) Foreign Application Priority Data
Mar. 17, 2021 (GB) 2103684.3

(51) Int. Cl. A61K 8/9789; A61K 8/27; A61K 8/49; A61Q 17/04

(52) U.S. Cl. CPC A61K 8/9789; A61K 8/27; A61K 8/4973; A61Q 17/04; A61K 2800/805

(57) ABSTRACT

The present invention provides a UV filter composition comprising coffee bean extract and Pongamia extract and a solar protection formulation comprising the UV filter composition.

The present invention relates to a UV filter composition and solar protection formulations comprising the UV filter composition. In particular, the present invention relates to a UV filter composition and a solar protection formulation comprising coffee bean extract extracted from recycled coffee grounds and Pongamia extract.

The damaging effects of sunlight on human skin are well known. Six percent of the solar energy reaching the Earth's surface is ultraviolet (UV) radiation having a wavelength of 280 to 400 nm. This radiation is divided into two components:
(i) low energy UVA radiation having a wavelength of 320 to 400 nm and
(ii) high energy UVB radiation having a wavelength of 280 to 320 nm.

While the UV portion of solar energy is relatively small, it induces nearly 99% of all the side effects from sunlight exposure. High energy UVB radiation is responsible for producing tanning, sunburn, erythema and skin cancer. Low energy UVA radiation penetrates deeper into the skin and causes long-term damage, DNA damage, ageing, as well as wrinkle formation.

By avoiding direct exposure to sunlight, individuals can avoid the serious effects caused by exposure to UV radiation. However, because of the nature of their work, it is challenging for some people to avoid such exposure. In addition, some people voluntarily expose their skin to the sun, e.g. to tan. Therefore, protection against the harmful effects of the sun is important.

The effectiveness of a given solar protection formulation, mainly against UVB radiation, is assessed by how well it protects the skin in terms of a Sun Protection Factor (SPF), which is defined as the ratio of the amount of energy required to produce a minimal erythema on sun protected skin to the amount of energy required to produce the same level of erythema on unprotected skin. The effectiveness of a solar protection formulation against UVA radiation is assessed by its Protector Factor UVA (PFA or PF UVA). These assessments may be carried out either in vivo, for example by using the COLIPA test (COLIPA. Sun Protection Factor Test Method; COLIPA: Bruxelles, Belgium, 1994) or in vitro, using spectrophotometric methods.

In vitro testing determines the amount of protection a solar protection formulation offers by spreading the solar protection formulation onto a quartz, acrylic, or plastic (PMMA, polymethylmethacrylate) slide and measuring how much UV radiation passes through the slide. The less radiation that passes through the slide, the higher the protection factor. Examples of such methods are described in Pissavini et al. J. Soc. Cosmet. Chem. 1989, 40, 127-133, EP1291640A1 and Cole et al. Photodermatol Photoimmunol Photomed. 2019, 35, 436-441, the disclosures of which are herewith incorporated by reference in this respect.

Protection from the harmful effects of UV radiation exposure is available in the form of topically applied formulations containing UV filters. Approval by a regulatory agency is generally required for inclusion of active agents as UV filters in formulations intended for human use.

Most UV filters used in solar protection products can be classified as mineral UV filters or organic UV filters.

Examples of suitable mineral UV filters include zinc-based UV filters, such as zinc oxide (ZnO), and titanium-based UV filters, such as titanium dioxide (TiO₂).

Examples of active agents which have been or are currently approved in various countries for use as organic UV filters are para-aminobenzoic acid (PABA), octyl dimethyl PABA (Padimate O), phenylbenzimidazole sulfonic acid, 2-ethoxyethyl p-methoxycinnamate (Cinoxate), dioxybenzone (Benzophenone-8), (2-hydroxy-4-methoxyphenyl)-phenylmethanone (Oxybenzone or Benzophenone-3), 3,3,5-trimethylcyclohexyl 2-hydroxybenzoate (Homosalate), menthyl anthranilate, 2-ethylhexyl-2-cyano-3,3-diphenylacrylate (Octocrylene), octyl methoxycinnamate (Octinoxate), ethyl hexyl salicylate (Octisalate), 4-hydroxy-2-methoxy-5-(oxo-phenylmethyl)benzene sulfonic acid (Sulisobenzone or Benzophenone-4), tris(2-hydroxyethyl)ammonium 2-hydroxybenzoate (Trolamine salicylate), terephthalylidene dicamphor sulfonic acid (Ecamsule), bis-ethylhexyloxyphenol methoxyphenyl triazine (BEMT or Bemotrizinol), ethylhexyl triazone, diethylhexyl butamido triazone (Iscotrizinol), methylene bis-benzotriazolyl tetramethylbutylphenol (Bisoctrizole), diethylamino hydroxybenzoyl hexyl benzoate, 4-tert-butyl-4′-methoxydibenzoylmethane (Avobenzone).

Most organic UV filters absorb high-energy ultraviolet radiation of a certain wavelength range, depending on their structure, and release the energy as lower-energy rays, thereby preventing the skin-damaging ultraviolet rays from reaching the skin. Most solar protection formulations, therefore, offer selective protection against certain UV wavelengths, depending on the absorption spectrum of the active ingredient(s) in the formulation.

However, for total protection against damage from the sun, the skin needs to be protected from both UVA and UVB radiation. Therefore, there is a need to provide “broad spectrum” solar protection formulations, which provide protection against both UVA and UVB radiation.

Furthermore, some of the common UV filters typically used in solar protection formulations reportedly have adverse toxicological or environmental effects, as well as negative sensory effects, which discourage people from using solar protection formulations. Therefore, it is desirable to employ natural and/or upcycled or recycled ingredients in a solar protection formulation, either as a substitute or to provide a booster effect to a UV active ingredient.

Coffee bean extracts have been used in solar protection formulations for a long time. Solar protection compositions, which contain all the soluble derivatives of coffee (i.e. both water soluble and organic solvent-soluble extracts), have been shown to absorb radiation in the whole of the ultraviolet spectrum; however these compositions are difficult to distribute over the skin, and their appearance makes them generally rather unsuitable for cosmetic use.

U.S. Pat. No. 4,793,990 describes that coffee bean extract obtained by extraction with an organic solvent from coffee grounds, which are the residues from the extraction of water-soluble constituents from roasted coffee (i.e. recycled coffee grounds), filters light selectively in the UVB wavelength range from 280 to 320 nm. The formulations exemplified therein contain more than 15% w/w coffee bean extract based on the total weight of the formulation and are capable of being uniformly distributed in cosmetic carriers suitable for forming a continuous film.

Pongamia extract is another natural extract and contains Pongamol as an active ingredient. Pongamia extract is a well-known natural UVA absorber (Reddy, M., Drug Invention Today, 2011, 3, 197-199).

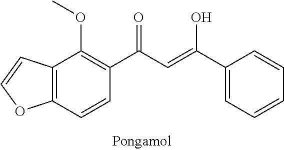 [SMILES: COc1c(C(=O)/C=C(\O)c2ccccc2)ccc2occc12]

There is still a need to provide UV filter compositions containing natural and/or upcycled or recycled ingredients, which show a good level of UV absorption in the whole range between 280 and 400 nanometers (nm), therefore providing both UVA and UVB protection against harmful solar radiation. There also remains a need for new solar protection formulations that provide high SPF and UVA rating while employing a minimum amount of UV filter agents.

### SUMMARY OF THE INVENTION

In a first aspect, the invention provides a UV filter composition comprising coffee bean extract and Pongamia extract.

In a further aspect, a solar protection formulation comprising a UV filter composition and a cosmetic base is provided, the UV filter composition comprising coffee bean extract and Pongamia extract.

The invention further provides a process for protecting human skin against UV radiation in the wavelength range of about 280 to about 400 nm comprising applying to the human skin to be protected a UV filter composition comprising coffee bean extract and Pongamia extract or a solar protection formulation comprising a UV filter composition comprising coffee bean extract and Pongamia extract and a cosmetic base.

In yet another aspect, use of a UV filter composition or a solar protection formulation comprising the UV filer composition for protecting an individual's skin from UV radiation in the wavelength range of about 280 to about 400 nm is provided, wherein the UV filter composition comprises coffee bean extract and Pongamia extract.

### BRIEF DESCRIPTION OF THE DRAWINGS

FIG. 1 shows an example of a process for the production of a coffee bean extract by supercritical CO₂ extraction of recycled coffee grounds;

FIG. 2 shows the UV absorption spectrum of a coffee bean extract obtained by supercritical CO₂ extraction of recycled coffee grounds;

FIG. 3 shows an example of a process for the production of a Pongamia extract;

FIG. 4 shows the UV absorption spectra recorded on PMMA plates of a Pongamia extract obtained by solvent extraction from Pongamia oil and crystallization in comparison with a blank sample;

FIG. 5 shows the UV absorption spectra recorded on PMMA plates of a coffee bean extract obtained by supercritical CO₂ extraction of recycled coffee grounds and of a combination of coffee bean extract and Pongamia extract;

FIG. 6 shows the UV absorption spectra recorded on PMMA plates of a solar protection formulation containing a zinc-based UV filter only;

FIG. 7 shows the UV absorption spectra recorded on PMMA plates of a solar protection formulation containing a zinc-based UV filter and a coffee bean extract;

FIG. 8 shows the UV absorption spectra recorded on PMMA plates of a solar protection formulation containing a zinc-based UV filter and Pongamia extract; and

FIG. 9 shows the UV absorption spectra recorded on PMMA plates of a solar protection formulation containing a zinc-based UV filter, a coffee bean extract and Pongamia extract.

### DETAILED DESCRIPTION

Preferred and/or optional features of the invention will now be set out. Any aspect of the invention may be combined with any other aspect of the invention unless the context demands otherwise. Any of the preferred or optional features of any aspect may be combined, singly or in combination, with any aspect of the invention, as well as with any other preferred or optional features, unless the context demands otherwise.

### UV Filter Compositions

Surprisingly, it was found that by combining two natural extracts, coffee bean extract and Pongamia extract, a synergistic increase (super booster effect) of both SPF and PF UVA was achieved. In particular, the boosting effect of the SPF and PF UVA of a mixture of coffee bean extract and Pongamia extract was not only much higher than the boosting effect of coffee bean extract or of Pongamia extract independently, but also much higher than the calculated sum of the two separate boosting effects.

The invention, therefore, provides a UV filter composition comprising coffee bean extract and Pongamia extract. The coffee beans may be sourced from Indonesia, Colombia, Brazil, Guatemala, Honduras, Ethiopia, Kenya, Nicaragua and Costa Rica and the Pongamia seeds from India, for instance.

### Coffee Bean Extract

The coffee bean extract may contain both water and organic solvent-soluble components. Alternatively, the coffee bean extract may contain only organic solvent-soluble components. The coffee bean extract may be obtained by any method known in the art that is suitable for extracting water and/or organic solvent-soluble components.

For example, the coffee bean extract may be obtained by step-wise extraction of the water and organic solvent-soluble components from coffee beans and combining the water and organic solvent extracts. In a first step, the coffee beans may undergo extraction with water, at one or more temperatures ranging from 0 to 120° C., under pressure ranging from 1 to 15 bar. The water extract may be separated from the residual coffee grounds by any method known in the art. The water extract may be optionally concentrated. The residual coffee grounds (recycled coffee grounds) may be optionally dried and further subjected to extraction using any suitable extraction method known in the art for extracting organic-soluble components. For example, the recycled coffee grounds may be subjected to extraction with an organic solvent, at one or more temperatures ranging from 0 to 150° C., depending on the solvent used. The organic solvent extract may be separated from the remaining coffee grounds by any method known in the art. The water extract and the organic extract may be combined and optionally concentrated using any suitable method known in the art, or each of the water and the organic extract may be optionally concentrated individually and the concentrated extracts combined.

Preferably, only the organic extract is used in the present invention.

Examples of suitable organic solvents include, but are not limited to, hydrocarbons, such as pentane, petroleum ether, hexane, heptane or cyclohexane; alcohols such as methanol, 2-methoxyethanol, ethanol, n-propanol, isopropanol, all isomers of butyl alcohol, benzyl alcohol, 1,2,6-trihydroxyhexane, ethylene glycol, 1,2-propanediol, dipropylene glycol, 1,3-butanediol, 2-butoxyethanol, 1,3-butylene glycol, glycerol; esters, such as ethyl acetate, iso-propyl acetate, 2-butoxyethyl acetate, glycerol esters such as glyceryl diacetate, glyceryl triacetate, glyceryl tributyrate; ethers, such as diethyl ether, 2-ethoxyethanol; ketones, such as acetone, 2-butanone; betaine, toluene, chlorinated or fluorinated hydrocarbons such as dichloromethane; and mixtures thereof, optionally with water, including eutectic mixtures.

In one embodiment, the coffee bean extract is obtained from recycled coffee grounds (i.e. coffee grounds which are the residues from the extraction of water-soluble constituents from roasted coffee). The spent coffee grounds are one of the major by-products in the world, reaching 9 billion kilograms per year. Using waste products to create new ones is clearly advantageous to the environment.

For example, the recycled coffee beans may be obtained by extraction of the water soluble components from coffee beans and discarding the water soluble extract. The coffee beans may undergo extraction with water, at one or more temperatures ranging from 0 to 120° C., under pressure ranging from 1 to 15 bar. The water extract may be separated from the residual coffee grounds by any method known in the art.

The residual coffee grounds (recycled coffee grounds) may be optionally dried and further subjected to extraction using any suitable extraction method known in the art for extracting organic-soluble components.

Any variety of coffee beans can be used, including Robusta and Arabica coffee beans, which are the two most widely used varieties in drinking coffee. In general, Robusta beans have a much higher content of caffeine than Arabica beans. On the other hand, a higher content of coffee oil, with a fatty acid composition of linoleic acid, palmitic acid, oleic acid, stearic acid, arachidic acid and linolenic acid can be generally found in Arabica coffee beans (about 15% w/w on a dry basis in Arabica coffee beans vs. about 10% w/w in Robusta coffee beans). Therefore, preferably, the coffee grounds are Arabica coffee grounds.

In one embodiment, the coffee bean extract is obtained by extraction with an organic solvent of the coffee bean grounds. For example, the coffee grounds may be subjected to extraction with an organic solvent, at one or more temperatures ranging from 0 to 150° C., depending on the solvent used. The organic solvent extract may be separated from the remaining coffee grounds by any method known in the art. The extract may be optionally concentrated using any suitable method known in the art.

In one embodiment of the invention, the coffee bean extract comprises coffee bean extract obtained by extraction with an organic solvent of recycled coffee grounds. For example, the recycled coffee grounds may be obtained as described above. The recycled coffee grounds are subsequently subjected to extraction with an organic solvent, at one or more temperatures ranging from 0 to 150° C., depending on the solvent used. The organic solvent extract may be separated from the remaining coffee grounds by any method known in the art. The organic extract may be optionally concentrated using any suitable method known in the art.

Suitable organic solvents are as defined above.

An example of a method for the preparation of a coffee bean extract obtained from recycled coffee grounds is described in U.S. Pat. No. 4,793,990, the disclosure of which is herewith incorporated by reference in this respect.

In one embodiment, the coffee bean extract is obtained by supercritical CO₂ extraction. For example, the coffee grounds may be subjected to extraction with supercritical CO₂.

Supercritical carbon dioxide is an attractive alternative to traditional organic solvents. Carbon dioxide behaves as a supercritical fluid above its critical pressure of 73.8 bar (1071 psi) and its critical temperature of 31.1° C. Under these conditions, it has the solvating power of a liquid and the diffusivity of a gas. This is advantageous because CO₂ can reach into small spaces like a gas but can also dissolve and act like a liquid solvent. This technology eliminates the use of organic solvents, which reduces the problems of their storage, disposal, and environmental concerns. Unlike many organic solvents, supercritical CO₂ is non-flammable. It is inert, non-toxic, has a relatively low cost and has moderate critical constants. Its solvation strength can be fine-tuned by adjusting the density of the fluid. CO₂ leaves a lower amount of residue in products compared to conventional solvents, and it is available in relatively pure form and in large quantities.

In one embodiment of the invention, the coffee bean extract comprises coffee bean extract obtained by supercritical CO₂ extraction of recycled coffee grounds.

For example, the recycled coffee grounds may be obtained as described above. The recycled coffee grounds are subsequently subjected to extraction using supercritical CO₂.

An example of a process for the production of a coffee bean extract by supercritical CO₂ extraction of recycled coffee grounds is shown in FIG. 1. The spent coffee grounds (i.e. the residual coffee grounds after the water soluble components have been removed, also referred to as recycled coffee grounds) are treated with alkaline water, then subjected to preservation, followed by drying and, optionally after being combined with industrial coffee waste, the mixture undergoes supercritical CO₂ extraction to yield coffee bean extract.

For instance, the recycled coffee bean extract is commercially available from Givaudan under the tradename Koffee'Up (https://www.givaudan.com/fragrance-beauty/active-beauty/products/koffee-up). The coffee bean extract obtained by supercritical CO₂ extraction of recycled coffee grounds is an oil rich in linoleic and palmitic acids, polyphenols, tocopherols and diterpene esters.

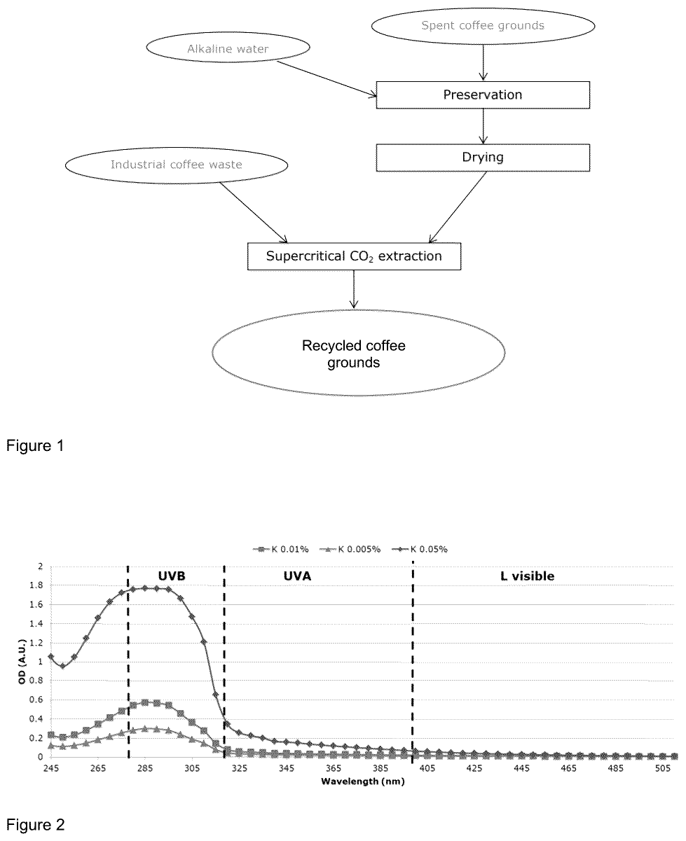

The UV absorption spectra of a coffee bean extract obtained by supercritical CO₂ extraction of recycled coffee grounds at increasing concentrations (0.005%, 0.01% and 0.05%) in ethanol absolute is shown in FIG. 2. The spectra confirm the ability of the coffee bean extract to absorb UV radiation in the UVB region.

### Pongamia Extract

Pongamia extract is derived from the seeds of the Milletia pinnata tree (also known as Indian Karanja or Pongamia glabra). Millettia pinnata has a number of different varieties, but little research has been published on the differences between them. This combined with variances in soil and weather can change the specific composition of the Pongamia oil, from which Pongamia extract is obtained. Pongamia oil can be obtained from the Pongamia seeds by expeller pressing, cold pressing, and/or solvent extraction.

In one embodiment of the invention, Pongamia seeds are crushed to obtain Pongamia (Karanja) oil. Pongamia oil is then subjected to solvent extraction. Subsequently, Pongamia extract enriched in Pongamol crystallizes from the solvent extract. Advantageously, by solvent extraction followed by isolation of the Pongamia extract by crude extract crystallization, Pongamia extract enriched in Pongamol (up to 90%) can be obtained. Optionally, further purification of the crystallized product by washing with a solvent and drying can yield further purified product.

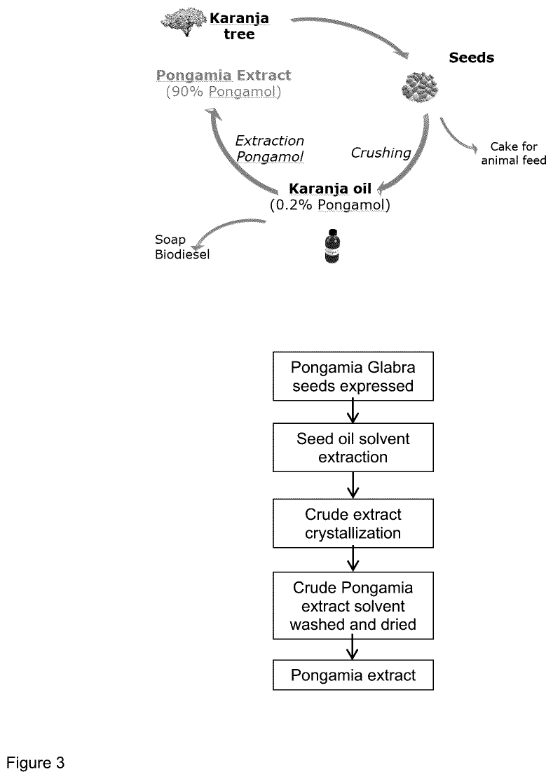

A suitable process for obtaining Pongamia extract is illustrated in FIG. 3.

Suitable solvents used for solvent extraction of Pongamia extract from Pongamia oil and crystallization include alcohols, such as methanol, ethanol and isopropanol.

For instance, the Pongamia extract refers to: https://www.qivaudan.com/fraqrance-beauty/active-beauty/products/ponqamia-extract.

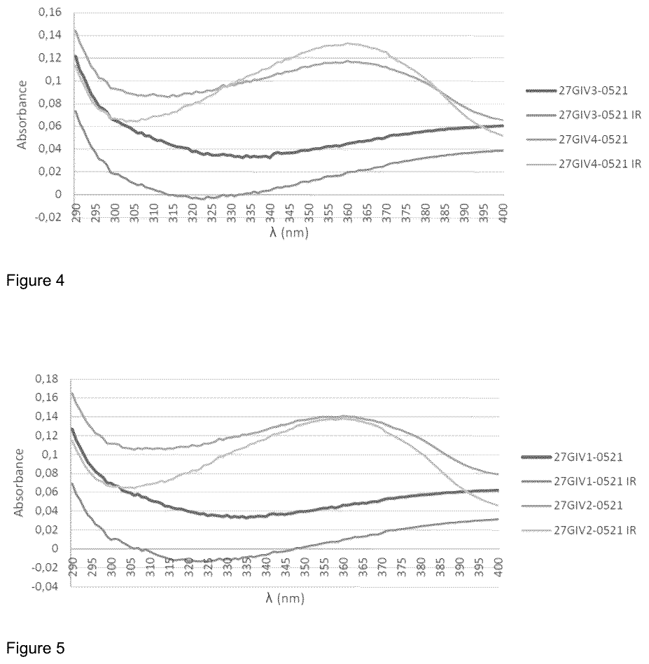

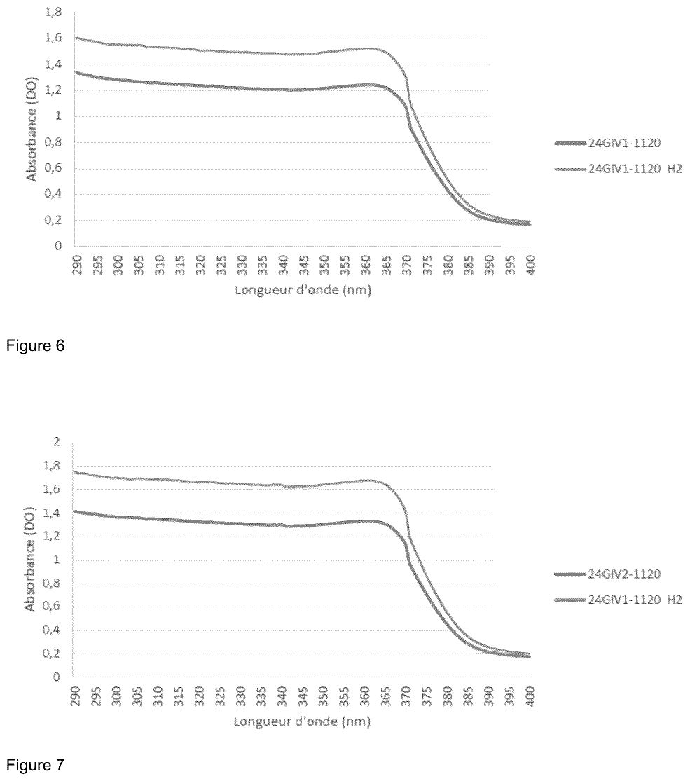

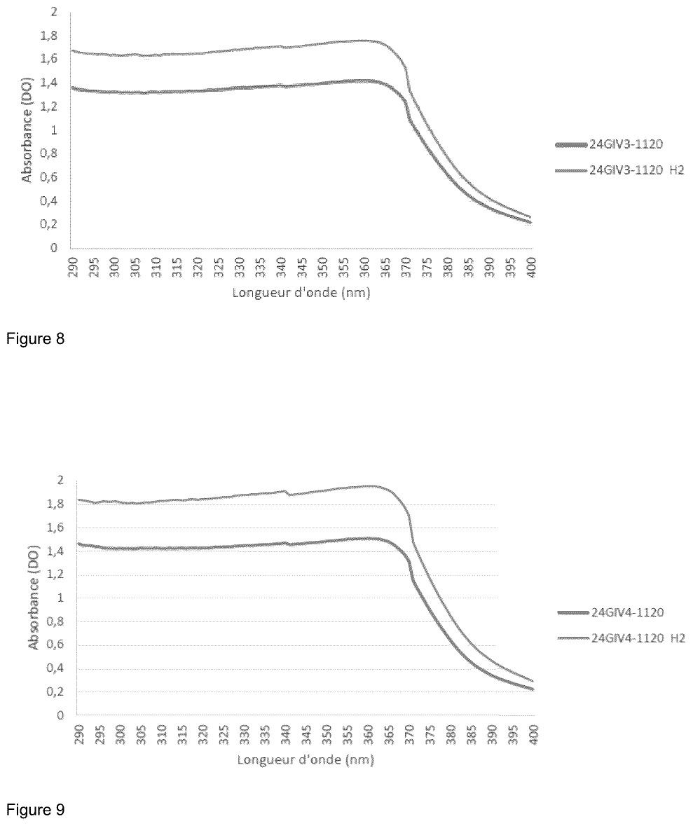

The UV absorption spectrum of Pongamia extract obtained by solvent extraction from Pongamia oil and crystallization was recorded by application on PMMA plates and measuring transmittance in the UV range of 290 nm to 400 nm in 1 nm steps (FIG. 4). The spectrum confirms the UV absorption capability of the Pongamia extract obtained by solvent extraction from Pongamia oil and crystallization in the UVA region of the UV spectrum.

### Coffee Bean Extract and Pongamia Extract

As has been discussed above, coffee bean extract mainly absorbs UV radiation in the UVB region, whereas Pongamia extract mainly absorbs UV radiation in the UVA region. Therefore, by combining the two extracts, it is possible to achieve effective UV protection over a broader wavelength range.

In addition, it was surprisingly observed that, by combining coffee bean extract with Pongamia extract as defined hereinbefore, the SPF and/or PF UVA value was vastly increased compared to the SPF and/or PF UVA value expected by simply adding the SPF and/or PF UVA values of the two individual components. Thus, there is clearly a synergistic booster effect.

Furthermore, the UV filter composition allows for the use of recycled coffee grounds, i.e. a waste stream product from the production of drinking coffee, thereby significantly increasing sustainability.

In one embodiment, the UV filter composition further comprises a mineral-based UV filter, wherein, preferably, the mineral-based UV filter can be a zinc-based UV filter, such as ZnO, or a titanium-based UV filter, such as TiO₂, or a combination thereof. More preferably the mineral-based UV filter is a zinc-based UV filter.

In one embodiment, the UV filter composition as hereinbefore described further comprises an organic-based UV filter, which can be selected from the group consisting of para-aminobenzoic acid (PABA), octyl dimethyl PABA (Padimate O), phenylbenzimidazole sulfonic acid, 2-ethoxyethyl p-methoxycinnamate (Cinoxate), dioxybenzone (Benzophenone-8), (2-hydroxy-4-methoxyphenyl)-phenylmethanone (Oxybenzone or Benzophenone-3), 3,3,5-trimethylcyclohexyl 2-hydroxybenzoate (Homosalate), menthyl anthranilate, 2-ethylhexyl-2-cyano-3,3-diphenylacrylate (Octocrylene), octyl methoxycinnamate (Octinoxate), ethylhexyl salicylate (Octisalate), 4-hydroxy-2-methoxy-5-(oxo-phenylmethyl)benzene sulfonic acid (Sulisobenzone or Benzophenone-4), tris(2-hydroxyethyl)ammonium 2-hydroxybenzoate (Trolamine salicylate), terephthalylidene dicamphor sulfonic acid (Ecamsule), bis-ethylhexyloxyphenol methoxyphenyl triazine (BEMT or Bemotrizinol), ethylhexyl triazone, diethylhexyl butamido triazone (Iscotrizinol), methylene bis-benzotriazolyl tetramethylbutylphenol (Bisoctrizole), diethylamino hydroxybenzoyl hexyl benzoate, 4-tert-butyl-4′-methoxydibenzoylmethane (Avobenzone) and any other suitable organic UV filter, or any combinations thereof.

However, as the list of approved mineral-based and organic-based UV filters is currently expanding, the skilled person will recognize that the invention is not limited to UV filters currently approved for human use but is readily applicable to those that may be allowed in the future.

### Solar Protection Formulations

Another aspect of the present invention provides a solar protection formulation comprising a UV filter composition as hereinbefore defined and a cosmetic base.

The cosmetic base may comprise any cosmetically acceptable excipient. A cosmetically acceptable excipient can be any excipient commonly used in the preparation of cosmetic preparations for use on the human skin. Suitable excipients include, but are not limited to, ingredients that can influence organoleptic properties and penetration of the skin, as well as the amount of time the active ingredient(s) remain active on the skin. More specifically, they include liquids, such as water, oils or surfactants, including those of petroleum, animal, plant or synthetic origin, such as and not restricted to, peanut oil, soybean oil, mineral oil, sesame oil, castor oil, polysorbates, sorbitan esters, ether sulfates, sulfates, betaines, glycosides, maltosides, fatty alcohols, nonoxynols, poloxamers, polyoxyethylenes, polyethylene glycols, dextrose, glycerol, digitonin, and the like, or combinations thereof.

In one embodiment, the solar protection formulation as defined hereinbefore is a solar cream, a solar lotion, a solar spray, a solar gel, a solar foam or a solar stick formulation.

In a preferred embodiment, the solar protection formulation is a solar cream formulation, optionally a face cream formulation. Creams are semi-solid emulsions of oil and water. Due to its both hydrophilic and lipophilic composition, a cream can incorporate a variety of active ingredients to be applied to an individual's skin.

The concentration of the coffee bean extract in a topical formulation can be up to about 0.30% w/w. In one embodiment, the solar protection formulation comprises about 0.10-0.30% w/w, preferably about 0.10%, 0.15%, 0.20%, 0.25% or 0.30% w/w, most preferably about 0.30% w/w coffee bean extract based on the total weight of the solar protection formulation.

The concentration of Pongamia extract in a topical formulation can be up to about 2.0% w/w. In one embodiment, the solar protection formulation comprises about 0.5-2.0% w/w, preferably about 1.0-2.0% w/w, preferably about 1.0%, 1.1%, 1.2%, 1.3%, 1.4%, 1.5%, 1.6%, 1.7%, 1.8%, 1.9% or 2.0% w/w, most preferably about 1.0% 1.5% or about 2.0% w/w Pongamia extract based on the total weight of the solar protection formulation.

In one embodiment, the solar protection formulation as defined hereinbefore further comprises a mineral-based UV filter, preferably a zinc-based UV filter, such as a ZnO-based UV filter.

In one embodiment, the solar protection formulation as defined hereinbefore further comprises an organic-based UV filter. Suitable organic-based UV filters include, but are not limited to, para-aminobenzoic acid (PABA), octyl dimethyl PABA (Padimate O), phenylbenzimidazole sulfonic acid, 2-ethoxyethyl p-methoxycinnamate (Cinoxate), dioxybenzone (Benzophenone-8), (2-hydroxy-4-methoxyphenyl)-phenylmethanone (Oxybenzone or Benzophenone-3), 3,3,5-trimethylcyclohexyl 2-hydroxybenzoate (Homosalate), menthyl anthranilate, 2-ethylhexyl-2-cyano-3,3-diphenylacrylate (Octocrylene), octyl methoxycinnamate (Octinoxate), ethylhexyl salicylate (Octisalate), 4-hydroxy-2-methoxy-5-(oxo-phenylmethyl)benzene sulfonic acid (Sulisobenzone or Benzophenone-4), tris(2-hydroxyethyl)ammonium 2-hydroxybenzoate (Trolamine salicylate), terephthalylidene dicamphor sulfonic acid (Ecamsule), bis-ethylhexyloxyphenol methoxyphenyl triazine (BEMT or Bemotrizinol), ethylhexyl triazone, diethylhexyl butamido triazone (Iscotrizinol), methylene bis-benzotriazolyl tetramethylbutylphenol (Bisoctrizole), diethylamino hydroxybenzoyl hexyl benzoate, 4-tert-butyl-4′-methoxydibenzoylmethane (Avobenzone), or combinations thereof.

In one embodiment, the solar protection formulation comprises
a) coffee bean extract obtained by supercritical CO₂ extraction of recycled coffee grounds;
b) Pongamia extract obtained by solvent extraction from Pongamia oil and crystallization; and, optionally,
c) a zinc-based UV filter.

In one embodiment, the solar protection formulation comprises
a) about 0.3% w/w coffee bean extract obtained by supercritical CO₂ extraction of recycled coffee grounds, based on the total weight of the solar protection formulation;
b) about 1.0% w/w Pongamia extract obtained by solvent extraction from Pongamia oil and crystallization, based on the total weight of the solar protection formulation; and, optionally,
c) a zinc-based UV filter.

Surprisingly, it was found that a solar formulation comprising both coffee bean extract obtained by supercritical CO₂ extraction of recycled coffee grounds and Pongamia extract obtained by solvent extraction from Pongamia oil and crystallization in addition to a zinc-based UV filter shows a synergistic increase (super booster effect) of both SPF and PF UVA when compared to the formulations comprising a zinc-based UV filter only.

### Methods of Use

The invention further provides a process for protecting human skin against UV radiation in the wavelength range of about 280 to about 400 nm, comprising applying to the human skin to be protected a UV filter composition or a solar protection formulation as defined hereinbefore.

In yet another aspect, use of a UV filter composition or of a solar protection formulation as defined hereinbefore for protecting an individual's skin from UV radiation in the wavelength range of about 280 to about 400 nm is provided.

The present invention is further illustrated by means of the following non-limiting examples:

### Example 1: Process of Production of Coffee Bean Extract Obtained by Supercritical CO₂ Extraction of Recycled Coffee Grounds and UV Absorption Properties

Spent coffee grounds (i.e. the residual coffee grounds after the water soluble components have been removed, also referred to as recycled coffee grounds) were treated with alkaline water, then subjected to preservation, followed by drying and, after being combined with industrial coffee waste, the mixture was extracted with supercritical CO₂ to yield recycled coffee bean extract.

FIG. 1 shows a diagrammatic representation of this process.

FIG. 2 shows the UV absorption spectra of coffee bean extract obtained by supercritical CO₂ extraction of recycled coffee grounds at increasing concentrations in ethanol absolute. The coffee extract was diluted in ethanol absolute at 0.005%, 0.01% and 0.05% (v/v) and the UV absorption spectra of the resulting solutions were measured by a spectrophotometer in a 1 cm path length cuvette. This study confirms the UVB absorption capability of the active ingredients present in the coffee bean extract obtained by supercritical CO₂ extraction of recycled coffee grounds.

### Example 2: Process of Production of Ponqamia Extract

FIG. 3 shows an example process of production of the Pongamia extract from Pongamia glabra seeds by solvent extraction from Pongamia oil and crystallization. The active ingredient in this extract is Pongamol.

Pongamia seeds were crushed to obtain Pongamia (Karanja) oil. Pongamia oil was then subjected to solvent extraction. Subsequently, Pongamia extract enriched in Pongamol (90% w/w Pongamol) was crystallized from the solvent extract. Optionally, further purification of the crystallized product by washing with a solvent and drying, can yield further purified product.

### Example 3: UV Absorption Properties of Coffee Bean Extract Obtained as in Example 1, Pongamia Extract Obtained as in Example 2 and a Combination of Coffee Bean Extract and Pongamia Extract

FIG. 4 shows the UV absorption spectra recorded on PMMA plates of Pongamia extract obtained by solvent extraction from Pongamia oil and crystallization (sample 27GIV4) in comparison with a blank sample (sample 27GIV3). This study confirms that Pongamia extract mainly absorbs in the UVA region of the UV spectrum.

FIG. 5 shows the UV absorption spectra recorded on PMMA plates of a coffee bean extract obtained by supercritical CO₂ extraction of recycled coffee grounds (sample 27GIV1) and of a combination of coffee bean extract and Pongamia extract (sample 27GIV2). It can be observed that the coffee bean extract obtained by supercritical CO₂ extraction of recycled coffee grounds does not absorb in the UVA region of the UV spectrum, unlike the combination of coffee bean extract and Pongamia extract.

The above-mentioned UV spectra were recorded by application of a sample to be measured on PMMA plates and measuring transmittance in the UV range 290 nm to 400 nm in 1 nm steps.

The samples composition is as shown in Table 1.

TABLE 1
SPF and PF UVA measurements for coffee bean
extract, Pongamia extract and a combination
of coffee bean extract and Pongamia extract

| | | Measured | PF | Claimed |
|---|---|---|---|---|
| Entry | Sample | SPF ^{a} | UVA ^{a} | SPF |
| 1 | blank | 1.2 | 1.1 | 0 |
| 2 | coffee bean extract 0.3% | 2.2 | 1.4 | 0 |
| 3 | Pongamia extract 1% | 1.2 | 1.3 | 0 |
| 4 | coffee bean extract 0.3% + | 1.2 | 1.3 | 0 |
| | Pongamia extract 1% | | | |

^{a} measured after IR irradiation

SPF and PF UVA values were recorded using the same procedure as described below for the solar protection formulations (see Example 4 below). The SPF value calculated using equation (1) and the PF UVA value calculated using equation (2) for the four samples are shown in Table 1. As can be seen from the data, none of the samples provide UVB (SPF) or UVA (PF UVA) protection.

### Example 4: Booster Effect of Coffee Bean Extract, Pongamia Extract and Super-Booster Effect of Combination of Coffee Bean Extract and Pongamia Extract

In vitro measurements of the SPF and PF UVA values were carried out on four solar protection formulations comprising UV filter active ingredients.

The four solar protection formulations comprising UV filter active ingredients are shown in Table 2.

TABLE 2
Composition of solar protection formulations

| Formulation | UV filter active ingredients in the formulation ^{a} |
|---|---|
| 1 | a zinc-based UV filter only (36.8) |
| 2 | a zinc-based UV filter (36.8); and |
| | coffee bean extract obtained by supercritical CO₂ extraction |
| | of recycled coffee grounds (0.3) |
| 3 | a zinc-based UV filter (36.8); and |
| | Pongamia extract obtained by solvent extraction from |
| | Pongamia oil and crystallization (1.0) |
| 4 | a zinc-based UV filter (36.8); and |
| | coffee bean extract obtained by supercritical CO₂ extraction |
| | of recycled coffee grounds (0.3) |
| | Pongamia extract obtained by solvent extraction from |
| | Pongamia oil and crystallization (1.0) |

^{a} values in parentheses represent % w/w, based on the total weight of the solar protection formulation

UV-transmitting PMMA plates (50 mm×50 mm) with a 5 μm medium roughness were used as a substrate. A measured amount of sample was spotted evenly on the surface of the PMMA plate.

The weight of the sample was recorded before any evaporation occurred. Using light pressure, the sample was immediately spread over the whole plate surface to achieve homogeneous distribution at an application thickness of 1.2 mg/cm² (for the SPF measurement) or 0.75 mg/cm² (for the PF UVA measurement). The transmittance was measured in the UV range of 290 nm to 400 nm in 1 nm steps. Up to nine UV transmittance spectra were recorded for each sample at different locations on the plate.

The SPF value for each formulation was calculated based on the spectral transmittance, as reported by Diffey et al., J. Soc. Cosmet. Chem. 1989, 40, 127-133, the disclosure of which is herein incorporated by reference, using equation (1):

$$SPF = \frac{{\int }_{250}^{400} {E}_{λ} {s}_{λ , er} d λ}{{\int }_{200}^{400} {E}_{λ} {τ}_{λ} {s}_{λ , er} d λ} \qquad ( 1 )$$

wherein

E_{λ} is the spectral irradiance of terrestrial sunlight corresponding to COLIPA “SPF test method” or DIN 67501;

s_{λ,er} is the relative effectiveness of UV radiation at wavelength λ (nm) in producing delayed erythema in human skin (so-called “erythema action spectrum”) corresponding to CIE publication No. 90(1991) or DIN 5031-10; and

τ_{λ} represents the spectral transmittance of a sample.

The PF UVA value for each formulation was calculated using equation (2), according to EP 1 291 640 A1:

$${UVAPF}_{calculated} = \frac{{\int }_{320 nm}^{400 nm} E ( λ ) * S ( λ )}{{\int }_{320 nm}^{400 nm} E ( λ ) * S ( λ ) / {10}^{( {A}_{2} * C )}} \qquad ( 2 )$$

wherein

E(λ) is irradiance at wavelength λ of the light spectrum used;

C is a constant factor for the adjustment of the spectrum to the in-vivo measurement;

S(λ) is the effectiveness of a biological UVA endpoint at wavelength λ; and

A(λ) is the absorbance.

Table 3 shows the SPF and PF UVA measured in vitro for the four solar protection formulations 1-4 described in Table 2.

TABLE 3
SPF and PF UVA values measured for the
four solar protection formulations 1-4

| Formulation | Measured SPF ^{a} | PF UVA ^{a} | | Claimed SPF |
|---|---|---|---|---|
| 1 | 28.4 | 12.7 | | 25 |
| 2 | 37.7 (+9.3) | 15 | (+2.3) | 30 |
| 3 | 37.7 (+9.3) | 21.7 | (+9) | 30 |
| 4 | 55.2 (+26.8) | 28.3 | (+15.6) | 50 |

^{a} value in parentheses shows the difference between the value measured for the formulation compared to the value measured for formulation 1

It has been found that solar protection formulations comprising either
1) 0.3% w/w (based on the total weight of the solar protection formulation) coffee bean extract obtained by supercritical CO₂ extraction of recycled coffee grounds in addition to a zinc-based UV filter (formulation 2); or
2) 1.0% w/w (based on the total weight of the solar protection formulation) Pongamia extract obtained by solvent extraction from Pongamia oil and crystallization in addition to a zinc-based UV filter (formulation 3)

show higher SPF and PF UVA (booster effect) compared to solar protection formulations comprising a zinc-based UV filter only (formulation 1).

The differences between the measured SPF and PF UVA values of formulation 3 and the measured SPF and PF UVA values of formulation 1 (of 9.3 and 9, respectively) are much larger than what would be expected for simply adding to formulation 1 Pongamia extract obtained by solvent extraction from Pongamia oil and crystallization at 1% w/w loading. The measured SPF value of 2% w/w Pongamia extract formulation was 3.3 and the PF UVA value was 3.8 (see Example 2).

Surprisingly, the solar formulation comprising both coffee bean extract obtained by supercritical CO₂ extraction of recycled coffee grounds and Pongamia extract obtained by solvent extraction from Pongamia oil and crystallization in addition to the zinc-based UV filter (formulation 4) shows a synergistic increase (super booster effect) of both claimed SPF (increase from 25 to 50) and PF UVA (increase from 12.7 to 28.3) when compared to the formulation comprising the zinc-based UV filter only (formulation 1).

The full composition (% w/w) of the four formulations 1-4 is shown in Table 4.

TABLE 4
Composition (% w/w) of the formulation 1 to 4

| | | Formulation | | | |
|---|---|---|---|---|---|
| | Ingredient | 1 | 2 | 3 | 4 |
| A | Emulium ® Delta MB^{a} | 5 | 5 | 5 | 5 |
| | Refined apricot oil^{b} | 2 | 2 | 2 | 2 |
| | Emogreen L15^{c} | 5 | 5 | 5 | 5 |
| | Dub 810^{d} | 4.5 | 4.5 | 4.5 | 4.5 |
| | UV-CUT ZNO-68-CG^{e} | 36.8 | 36.8 | 36.8 | 36.8 |
| | Pongamia extract | — | — | 1 | 1 |
| | Koffee-Up | — | 0.3 | — | 0.3 |
| B | Water | 39.1 | 38.8 | 38.1 | 37.8 |
| | Avicel PC611^{f} | 1.2 | 1.2 | 1.2 | 1.2 |
| C | Keltrol KGSFT^{g} | 0.3 | 0.3 | 0.3 | 0.3 |
| D | Dermofeel GSC POF^{h} | 1 | 1 | 1 | 1 |
| E | Pentylene glycol | 5 | 5 | 5 | 5 |
| F | Dermofeel PA3ⁱ | 0.1 | 0.1 | 0.1 | 0.1 |

^{a}Supplied by Gattefossé
^{b}Supplied by Naturex
^{c}Supplied by Seppic
^{d}Supplied by Stearinerie-dubois
^{e}Supplied by Grant Industries, Inc.
^{f}Supplied by FMC Corporation
^{g}Supplied by CP Kelco
^{h}Supplied by Evonik
ⁱSupplied by Evonik Dr. Straetmans

1. A UV filter composition comprising coffee bean extract and Pongamia extract.

2. The UV filter composition according to claim 1, wherein the coffee bean extract is obtained from recycled coffee grounds.

3. The UV filter composition according to claim 1, wherein the coffee bean extract is obtained
a) by extraction with an organic solvent; and/or
b) by supercritical CO₂ extraction.

4. The UV filter composition according to claim 1, wherein the Pongamia extract is obtained by solvent extraction from Pongamia oil and crystallization, and, optionally purification.

5. The UV filter composition according to claim 1, further comprising a mineral-based UV filter.

6. The UV filter composition according to claim 1, further comprising an organic-based UV filter.

7. A solar protection formulation comprising the UV filter composition according to claim 1 and a cosmetic base.

8. The solar protection formulation according to claim 7, wherein the solar protection formulation is a solar cream, a solar lotion, a solar spray, a solar gel, a solar foam or a solar stick formulation.

9. The solar protection formulation according to claim 7, comprising about 0.10-0.30% w/w coffee bean extract based on the total weight of the solar protection formulation.

10. The solar protection formulation according to claim 7 comprising about 0.5-2.0% w/w Pongamia extract based on the total weight of the solar protection formulation.

11. The solar protection formulation according to claim 7 comprising
a) coffee bean extract obtained by supercritical CO₂ extraction of recycled coffee grounds;
b) Pongamia extract obtained by solvent extraction from Pongamia oil and crystallization; and, optionally,
c) a zinc-based UV filter.

12. A process for protecting human skin against UV radiation in the wavelength range of about 280 to about 400 nm, comprising applying to the human skin to be protected a UV filter composition according to claim 1.

13. (canceled)

14. The UV filter composition according to claim 2, wherein the coffee bean extract is obtained from recycled Arabica coffee grounds.

15. The UV filter composition according to claim 5, wherein the mineral-based UV filter is a zinc-based UV filter, a titanium-based UV filter, or a combination thereof.

16. The UV filter composition according to claim 6, wherein the organic-based UV filter is selected from the group consisting of para-aminobenzoic acid (PABA), octyl dimethyl PABA (Padimate O), phenylbenzimidazole sulfonic acid, 2-ethoxyethyl p-methoxycinnamate (Cinoxate), dioxybenzone (Benzophenone-8), (2-hydroxy-4-methoxyphenyl)-phenylmethanone (Oxybenzone or Benzophenone-3), 3,3,5-trimethylcyclohexyl 2-hydroxybenzoate (Homosalate), menthyl anthranilate, 2-ethylhexyl-2-cyano-3,3-diphenylacrylate (Octocrylene), octyl methoxycinnamate (Octinoxate), ethylhexyl salicylate (Octisalate), 4-hydroxy-2-methoxy-5-(oxo-phenylmethyl)benzene sulfonic acid (Sulisobenzone or Benzophenone-4), tris(2-hydroxyethyl)ammonium 2-hydroxybenzoate (Trolamine salicylate), terephthalylidene dicamphor sulfonic acid (Ecamsule), bis-ethylhexyloxyphenol methoxyphenyl triazine (BEMT or Bemotrizinol), ethylhexyl triazone, diethylhexyl butamido triazone (Iscotrizinol), methylene bis-benzotriazolyl tetramethylbutylphenol (Bisoctrizole), diethylamino hydroxybenzoyl hexyl benzoate, 4-tert-butyl-4′-methoxydibenzoylmethane (Avobenzone), and combinations thereof.
